(19) United States
(12) Patent Application Publication
(10) Pub. No.: US 2020/0077936 A1
(43) Pub. Date: Mar. 12, 2020

(54) SWEAT SENSING DEVICE KIDNEY BIOMARKER MEASUREMENT

(71) Applicant: Eccrine Systems, Inc., Cincinnati, OH (US)

(72) Inventors: Kathy A Cunningham, Cincinnati, OH (US); Benjamin Katchman, Tucson, AZ (US); Gavi Begtrup, Cincinnati, OH (US)

(73) Assignee: Eccrine Systems, Inc., Cincinnati, OH (US)

(21) Appl. No.: 16/469,940

(22) PCT Filed: Dec. 14, 2017
(86) PCT No.: PCT/US17/66415

§ 371 Date: Jun. 14, 2019

Related U.S. Application Data
(60) Provisional application No. 62/434,102, filed on Dec. 14, 2016.

(51) Int. Cl. A61B 5/20; A61B 5/145; A61B 5/00; A61B 5/01; A61B 5/053

(52) U.S. Cl. CPC A61B 5/201; A61B 5/14517; A61B 5/14539; A61B 2562/0219; A61B 5/4266; A61B 5/01; A61B 5/0533; A61B 5/14546

(57) ABSTRACT

The present disclosure provides a method of using a sweat sensing device to determine a kidney profile for an individual. The method includes taking concentration, ratio, and trend measurements of one or more kidney biomarkers in the individual's sweat, along with other contemporaneous device measurements to inform sweat rate, skin temperature, sweat sample pH, or other factors. The method further considers these measured values in the context of external information about the individual, and uses such information to develop a kidney function profile or a kidney injury profile indicative of the physiological state of the individual.

### CROSS-REFERENCE TO RELATED APPLICATIONS

This application claims priority to PCT/US17/66415, filed Dec. 14, 2017, and U.S. Provisional Application No. 62/434,102, filed Dec. 14, 2016, and has specification that builds upon PCT/US16/36038, filed Jun. 6, 2016, the disclosures of which are incorporated herein by reference in their entirety.

### BACKGROUND OF THE INVENTION

Sweat sensing technologies have enormous potential for applications ranging from athletics, to neonatology, pharmacological monitoring, and personal digital health. The available applications for sweat sensing technologies are so numerous because sweat contains many of the same biomarkers, chemicals, and solutes that are carried in blood. The presence of these biomarkers, chemicals, and solutes in sweat can provide significant information for non-invasively diagnosing ailments, health status, toxins, performance, and other physiological attributes, even in advance of any physical sign. Furthermore, sweat itself, and the action of sweating, as well as other parameters, attributes, solutes, or features on or near skin or beneath the skin, can be measured to further reveal physiological information.

Among the biofluids used for physiological monitoring (e.g., blood, urine, saliva, tears), sweat has arguably the least predictable sampling rate in the absence of technological solutions. An excellent summary of the challenges in sweat sampling is provided by Sonner, et al. in the 2015 article titled “The microfluidics of the eccrine sweat gland, including biomarker partitioning, transport, and biosensing implications,” Biomicrofluidics 9, 031301, herein included by reference in its entirety. With proper application of technology, however, sweat can be made to outperform other non-invasive or less invasive biofluids in predictable sampling. In particular, sweat sensing devices hold tremendous promise for use in workplace safety, athletic, military, and clinical diagnostic settings.

An important aspect of predictable sweat sampling is providing decision support that is informative at the level of the individual user. A sweat sensing device worn on the skin and connected to a computer network via a reader device, such as a smart phone or other portable or stationary device, can aid in recognition of the physiological state of the wearer; and relay crucial data that can inform decision-making about medical treatment, physical training, safety requirements, and other applications. Sweat sensors have the potential to continuously monitor one or more aspects of an individual's physiological state. Relevant information of the wearer's physiological state can then be communicated to a computer network and compared to threshold readings. From this comparison, notification messages can be generated and communicated to the individual, a caregiver, a work supervisor, or other device user.

Kidney biomarker concentrations, ratios, or trends can provide valuable information regarding a number of physiological states for an individual, and inform ongoing therapeutic decision making. Accordingly, it is desirable to have methods of non-invasively measuring one or more kidney biomarkers using a wearable sweat sensing device. In particular, it is desirable to measure one or more kidney biomarkers in sweat using a wearable sweat sensing device, and use the kidney biomarker measurements to develop a kidney function profile and/or a kidney injury profile. Specifically, it is desirable to have devices and methods for measuring and indicating an individual's sweat concentrations of kidney biomarkers, and interpreting those concentrations, ratios, and trends to inform a health condition for the individual.

### SUMMARY OF THE INVENTION

The present disclosure provides methods for using a sweat sensing device to measure kidney biomarker levels in sweat, and for determining from the biomarker measurements one or more physiological states experienced by a device wearer. In particular, in the methods described herein, the measured biomarker levels can be compared to one or more baseline levels to inform whether a device wearer is experiencing a kidney-related physiological condition. In a first embodiment, the method includes taking concentration, ratio, and trend measurements of one or more kidney biomarkers using a sweat sensing device, along with contemporaneous other device measurements to inform sweat rate, skin temperature, sweat sample pH, or other factors. The method further considers these measured values in the context of external information about the individual, and uses such information to develop a kidney function profile or a kidney injury profile which is indicative of a physiological state of the individual. Results are then communicated to a device user. Also included is a method of using a sweat sensing device to determine whether an individual has a physiological condition by comparing device measurements to a baseline kidney profile for the individual or individual's phenotype. The present invention is premised on the realization that sweat can be effectively analyzed in a single, continuous, or repeated manner inside the same device, and addresses applications of a sweat sensing device based on such capabilities to diagnose kidney-related health conditions.

### BRIEF DESCRIPTION OF THE DRAWINGS

The present disclosure will be further appreciated in light of the accompanying drawing figures in which:

FIG. 1 is a schematic representation of an exemplary sweat sensing system;

FIG. 2 is a schematic depiction, in cross-section, of an exemplary sweat sensing device;

FIG. 3 is an exemplary flow chart depicting a method of indicating an individual's kidney function profile in sweat; and

FIG. 4 is an exemplary flow chart depicting a method of indicating an individual's kidney injury profile in sweat.

### DEFINITIONS

Before continuing with a detailed description of the exemplary embodiments, a variety of definitions should be made, these definitions gaining further appreciation and scope in the detailed description and embodiments of the present disclosure.

As used herein, “sweat” means a biofluid that is primarily sweat, such as eccrine or apocrine sweat, and may also include mixtures of biofluids such as sweat and blood, or sweat and interstitial fluid, so long as advective transport of the biofluid mixtures (e.g., flow) is primarily driven by sweat.

“Sweat sensor” means any type of sensor that measures a state, presence, flow rate, solute concentration, solute presence, in absolute, relative, trending, or other ways in sweat. Sweat sensors can include, for example, potentiometric, amperometric, impedance, optical, mechanical, antibody, peptide, aptamer, or other means known by those skilled in the art of sensing or biosensing.

“Analyte” means a substance, molecule, ion, or other material that is measured by a sweat sensing device.

“Measured” can imply an exact or precise quantitative measurement and can include broader meanings such as, for example, measuring a relative amount of change of something. Measured can also imply a binary measurement, such as ‘yes’ or ‘no’ type measurements.

“Chronological assurance” means the sampling rate or sampling interval that assures measurement(s) of analytes in sweat in terms of the rate at which measurements can be made of new sweat analytes emerging from the body. Chronological assurance may also include a determination of the effect of sensor function, potential contamination with previously generated analytes, other fluids, or other measurement contamination sources for the measurement(s). Chronological assurance may have an offset for time delays in the body (e.g., a well-known 5 to 30 minute lag time between analytes in blood emerging in interstitial fluid), but the resulting sampling interval (defined below) is independent of lag time, and furthermore, this lag time is inside the body, and therefore, for chronological assurance as defined above and interpreted herein, this lag time does not apply.

“EAB sensor” means an electrochemical aptamer-based biosensor that is configured with multiple aptamer sensing elements that, in the presence of a target analyte in a fluid sample, produce a signal indicating analyte capture, and which signal can be added to the signals of other such sensing elements, so that a signal threshold may be reached that indicates the presence of the target analyte. Such sensors can be in the form disclosed in U.S. Pat. Nos. 7,803,542 and 8,003,374 (the “Multi-capture Aptamer Sensor” (MCAS)), or in U.S. Provisional Application No. 62/523,835 (the “Docked Aptamer Sensor” (DAS)).

“Analyte-specific sensor” means a sensor specific to an analyte which performs specific chemical recognition of the analyte's presence or concentration (e.g., ion-selective electrodes, enzymatic sensors, electro-chemical aptamer based sensors, etc.). For example, sensors that sense impedance or conductance of a fluid, such as sweat, are excluded from the definition of “analyte-specific sensor” because sensing impedance or conductance merges measurements of all ions in sweat (i.e., the sensor is not chemically selective; it provides an indirect measurement). Sensors can also be optical, mechanical, or use other physical/chemical methods which are specific to a single analyte. Further, multiple sensors can each be specific to one of multiple analytes.

“Sweat sensor data” means all of the information collected by sweat system sensor(s) and communicated via the system to a user or a data aggregation location.

“Correlated aggregated sweat sensor data” means sweat sensor data that has been collected in a data aggregation location and correlated with outside information such as time, temperature, weather, location, user profile, other sweat sensor data, or any other relevant data.

“Galvanic skin response” (GSR) means measurements of the electrical conductivity of the skin. GSR serves as a means of estimating sweat rate, since skin conductivity is dominated by the contribution of sweat, and increases linearly with increases in sweat rate throughout the linear range of 0.4 μL/cm²/min to 1.5 μL/cm²/min.

“Sweat conductivity” means measurements of the electrical conductivity of sweat. Sweat conductivity serves as a means of estimating Cl⁻ content, since Cl⁻ represents the dominant anion in sweat. However, conductivity does not precisely correlate to Cl⁻ levels, because lactate and bicarbonate also make significant contributions to sweat conductivity. A sweat sensing device as described herein would measure sweat conductivity by means of an electrode.

“Sensor response lag” means the difference between the point in time that a sweat sample reaches an analyte-specific sensor and the point at which the sensor produces an electrical signal corresponding to the sweat sample.

“Kidney function profile” means the concentrations, trends or ratios of relevant kidney biomarkers in sweat that reflect an individual's degree of kidney function. The kidney function profile may be compared to an individual's, or a phenotypical group's, “normal” or “baseline” level of circulating kidney biomarkers that indicate a reference degree of kidney function for the individual or group. Such a profile may include other relevant sweat sensor data, or external data.

“Kidney injury profile” means the concentrations, trends or ratios of relevant kidney biomarkers in sweat that reflect whether, or to what extent, an individual has sustained a kidney injury. Such a profile may include other relevant sweat sensor data, or external data.

### DETAILED DESCRIPTION OF THE INVENTION

The embodiments described herein will be primarily, but not entirely, limited to wearable sweat sensing devices, and methods or sub-methods using wearable sweat sensing devices. The disclosed embodiments may be practiced using any type of wearable sweat sensing device that measures sweat, sweat generation rate, sweat chronological assurance, sweat solutes, solutes that transfer into sweat from skin, a property of, or things on, the surface of skin, or properties or things beneath the skin. A sweat sensing device as discussed herein can take on many forms, including patches, bands, straps, portions of clothing or equipment, or any suitable mechanism that reliably brings sweat stimulating, sweat collecting, and/or sweat sensing technology into intimate proximity with sweat as it is generated. Applicable sweat sensing technology is described in the article titled “Adhesive RFID Sensor Patch for Monitoring of Sweat Electrolytes”, published in the journal IEEE Transactions on Biomedical Engineering; the article published in the journal AIP Biomicrofluidics, 9 031301 (2015), titled “The Microfluidics of the Eccrine Sweat Gland, Including Biomarker Partitioning, Transport, and Biosensing Implications”; as well as U.S. Provisional Application No. 62/064,009 and U.S. Provisional Application No. 62/120,342; each of which is included herein by reference in its entirety.

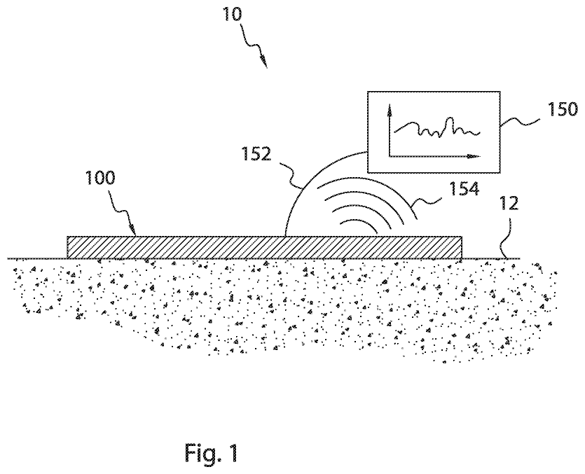

Turning now to FIG. 1, which depicts a representative sweat sensing system 10 to which the present disclosure applies. The sweat sensing system 10 includes a sweat sensing device 100 placed on or near skin 12. The sweat sensing device 100 may be fluidically connected to skin 12, or regions near the skin, through microfluidics or other suitable techniques. Device 100 is in wired communication 152 or wireless communication 154 with a reader device 150, which can be a smart phone or other portable electronic device or, for some embodiments, the sensing device 100 and reader device 150 can be combined. Communication 152 or 154 is preferably not continuous, and could occur periodically, at set or variable time periods, or as a simple, one-time data download from the sensing device 100 to the reader device 150 once the sensing device has completed sweat measurements.

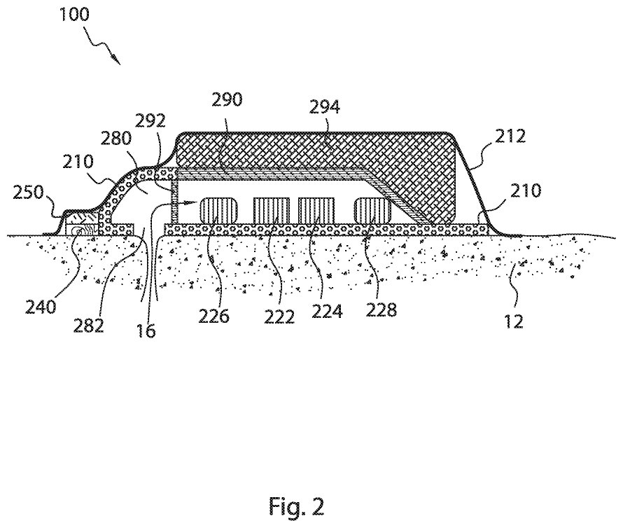

FIG. 2 depicts a more detailed, cross-sectional view of a representative sweat sensing device 100. As shown in FIG. 2, the device 100 includes a water-impermeable substrate 210, a protective covering 212, a microfluidic channel 280, an inlet 282, and a sweat collector (not shown) to introduce a sweat sample into the device. The substrate 210 is constructed of PET, Kapton, or PVC, and the cover 212 may be water impermeable rigid plastic, or in some embodiments, a breathable, waterproof fabric. The channel 280 is configured to concentrate a sweat sample relative to a target analyte, and includes an optional pre-concentration filter 292, a selectively-permeable concentrator membrane 290 and a concentrator pump 294. Such devices are disclosed in more detail in PCT/US16/58356, filed Oct. 23, 2016, which is incorporated herein by reference in its entirety.

When a sweat sample enters the channel 280 through the inlet 282, the sample moves in the direction of the arrow 16, where it encounters the pre-filter 292, which could be a track-etched membrane, a cellulose triacetate filter, Dow Filmtec™, or other suitable material. The filter 292 removes solutes from the sweat sample based on size, electrical charge, or chemical property, or removes proteases or other solutes that may interfere with the device measurements. Once through the filter 292, the sweat sample is concentrated relative to the target analyte by the concentrator membrane 290, which could be, for example, a dialysis membrane, or other material that at least allows the passage of water and inorganic solutes, but prevents passage of the target analyte. Depending on the application, the target analyte may be concentrated to a level at least 10×, 100×, or 1000× higher than the unconcentrated molarity. The pump 294 is constructed of a desiccant, a wicking hydrogel, paper, fabric, or other material suitable for drawing water out of the channel through the membrane.

As the sweat sample moves through the channel 280, it becomes increasingly concentrated, and interacts with at least one analyte-specific sensor 222, 224 and at least one secondary sensor 226, 228. The analyte-specific sensor(s) 222, 224 are EAB or enzymatic sensors for one or more kidney biomarkers. The secondary sensor(s) 226, 228 may include a micro-thermal flow rate sensor, one or more ISEs for measuring electrolytes (pH, Na⁺, Cl⁻, K⁺, Mg²⁺, etc.), a sweat conductivity sensor, a temperature sensor, or other sensor. Some embodiments may also include a sweat stimulant gel 240 composed of a sweat stimulant, such as carbachol or pilocarpine, and agar, and an iontophoresis electrode 250. The electrode 250 and stimulant gel 240 provide iontophoretic sweat stimulation as needed. Once sweat is stimulated, the electrode 250 can also be used to measure skin impedance or galvanic skin response (“GSR”), which indicates sweat onset or sweat cessation timing.

Embodiments of the sweat sensing device 100 may include a plurality of additional components or sensors to improve detection of sweat analytes, including a reference electrode, a pH sensor, a temperature sensor, a galvanic skin response sensor, a sweat conductivity sensor, a skin impedance sensor, a capacitive skin proximity sensor, a sweat rate sensor, and an accelerometer. The sweat sensing device 100 may also include computing and data storage capability sufficient to operate the device, such as the ability to conduct communication among system components, perform data aggregation, and execute algorithms capable of generating notification messages. The sweat sensing device may have varying degrees of onboard computing capability (i.e., processing and data storage capacity). For example, all computing resources could be located onboard the device, or some computing resources could be located on a disposable portion of the device and additional processing capability located on a reusable portion of the device. Alternatively, the device may rely on portable, fixed or cloud-based computing resources. In addition to the above, sweat sensing devices and systems as described herein may contain other aspects including, without limitation, two or more counter electrodes, reference electrodes, an onboard real-time clock, an onboard flash memory (i.e., 1 MB minimum), Bluetooth™ or other communications hardware, a multiplexer to process a plurality of sensor outputs, and additional supporting technology or features which are not captured in the description herein, but would be otherwise known to those skilled in the art.

The data aggregation capability of the device 100 may include collecting all of the sweat sensor data either generated by the device or communicated to the device. The aggregated sweat sensor data could be de-identified from individual wearers, or could remain associated with an individual wearer. The sweat sensor data can also be correlated with external, non-sweat factors, such as the time, date, time of day, medications, drug sensitivity, medical condition, activity performed by the individual, motion level, fitness level, mental and physical performance during the data collection, body orientation, the proximity to significant health events or stressors, age, sex, health history, or other relevant information. The reader device 150 can also be configured to correlate speed, location, environmental temperature, or other relevant data with the sweat sensor data communicated from device 100. The aggregated data can be made accessible via a secure website portal to allow sweat system users to perform safety, compliance and/or care monitoring of target individuals. The sweat sensor data that may be monitored includes real-time data and trend data; as well as aggregated sweat sensor data drawn from a system database and correlated to a particular individual, individual profile (such as age, sex or fitness level), weather condition, activity, combined analyte profile, or another relevant metric. Trend data, such as, for example, a target individual's hydration level over time, can be used to predict future performance, or the likelihood of an impending physiological event. Such predictive capability can be enhanced by using correlated aggregated data, which would allow a system user to compare an individual's historical analyte and external data profiles, to a real-time situation as it progresses, or even to compare thousands of similar analyte and external data profiles from other individuals to the real-time situation. Sweat sensor data may also be used to identify individuals that are in need of additional monitoring or instruction, such as the need to drink additional water, or to adhere to a drug regimen.

Because the sweat sensing device 100 could produce potentially sensitive physiological data, some database fields may be routinely encrypted. A preferred encryption method is the Advanced Encryption Standard. The device 100 will access a random 128-bit encryption and decryption key that will be generated and stored by a companion reader device 150 when needed for data transmission. In addition, because some sweat sensor data may repeat frequently, additional protection will be provided by introducing a random initialization vector before the encryption of each value. This will prevent observable patterns from emerging in the encrypted sweat sensor data. Other encryption methods and steps may be required, and applied according to best practices, as known to those skilled in the art.

Determining an individual's physiological state is a significant challenge. Not only is every individual different in terms of how a particular physiological state may present, but even a simple physiological state or disorder can be a complex set of biological processes that does not readily lend itself to reduction. Consequently, a definitive diagnosis of a physiological condition often is not possible. One solution to this problem is to classify individuals according to phenotypes or susceptibilities that indicate the mode in which a physiological state is likely to manifest in those individuals. These phenotypes may be indicated by characteristic analyte signatures that emerge in sweat.

Sweat is known to contain a large number of molecules and compounds that could be used to indicate an individual's physiological state. Among the most common substances found in sweat are the following: Na⁺, Cl⁻, K⁺, Ammonium (NH₄⁺), urea, lactate, glucose, serine, glycerol, cortisol, and pyruvate. In addition to these common sweat analytes, each physiological condition may also have particular sweat analytes that will prove informative for indicating that condition. For example, blood creatinine levels have proven useful for indicating hydration levels, and these same creatinine levels may be present as a hydration indicator in sweat.

To date, there have been relatively few studies linking sweat analytes to physiological states, with the existing studies linking increased sweat chloride levels with cystic fibrosis, or a spike in chloride levels with ovulation. Therefore, in order to establish a relevant baseline for diagnosing physiological conditions using sweat analytes, it likely is necessary to build a reference data profile across multiple individuals correlating one or more physiological states with characteristic sweat analyte levels for informing or diagnosing the state. Development of a reference profile may inform which analyte signatures indicate which physiological states, in each of a number of different phenotypes. The translation of sweat analyte concentrations and ratios to meaningful physiological information should take into account a number of variabilities unrelated to differences in concentrations. For example, the concentrations of analytes in sweat, as compared to the concentrations in blood or plasma, is known to vary depending on sweat rate, the body location from which a sample is taken, kidney or liver disease or function, external temperatures, and other factors. In developing meaningful physiological information, therefore, it will be necessary to use algorithms and techniques that reflect how the various analyte measurements change in response to these variabilities, and what effect changes have on the physiological state of an individual or phenotype.

Kidney biomarker concentrations, ratios, or trends can provide insight into a number of different physiological conditions. Blood levels of different biomarkers, such as neutrophil gelatinase-associated lipocalin (NGAL), vary in response to kidney function, as well as kidney injury. Some of the same biomarkers found in blood, including NGAL, also emerge in sweat. Thus, the non-invasive measurement of different kidney biomarkers in sweat may provide valuable insight into kidney function before various conditions manifest symptomatically. One common metric of kidney function is glomerular filtration rate (GFR), which is a measure of the flow rate of fluid through the kidney as the fluid is filtered by the nephrons, the kidney's basic functional unit. The nephron's proximal tubule and thick ascending limb are highly metabolically active, meaning they continuously require large amounts of oxygen and fuel to properly function. Interruptions of oxygen or fuel to the nephrons leads to a mixture of cell injury and death. Successful repair of injured cells is key to preservation of GFR, and is the reason that prompt medical intervention is so important to the maintenance of proper kidney function. If kidney injury is not promptly reversed, a downward spiral may be established in which nephron injury is followed by nephron death, which results in reduced GFR, which causes higher metabolic and functional demands on the remaining healthy nephrons, which causes increased sensitivity to further nephron injury. While a delay or failure of kidney tissue repair leads to further kidney damage and chronic disease, timely tissue repair can greatly contribute to the recovery of kidney function.

Inhibiting the timely treatment of kidney injury is the inability to directly measure GFR non-invasively. In the absence of reliable, non-invasive means to monitor kidney injury biomarkers, medical practitioners have relied on indirect measures of kidney function, such as plasma levels of urea or creatinine. For example, the creatinine clearance rate (C_{cr}), which is the volume of plasma that is filtered of creatinine per unit time, is commonly used. Another metric is serum creatinine concentration (sCr). However, sCr, like other indirect metrics, may vary widely with differences in age, sex, diet, muscle mass, hydration level, and other factors. Further, sCr lags deficiencies in kidney function by several hours or days, and deficiencies are often not apparent until the kidneys have lost as much as 50% of their function. See Devarajan, P., “Neutrophil gelatinase-associated lipocalin (NGAL): A new marker of kidney disease,” Scandinavian J. Clin. Lab. Investigation Supplemental, 2008; 241: 89-94. Accordingly, biomarkers capable of non-invasive and timely indications of kidney function and injury are needed to provide faster assessments of kidney function than creatinine, and to cover the considerable range of function loss prior to the 50% threshold. Specific biomarkers may also provide much needed information about the primary injury location within the kidney, the severity and duration of the injury, as well as the type of injury.

Kidney injury can generally be grouped into one of the following categories: oxygen deprivation, toxicity, inflammation, and trauma. Oxygen deprivation has a number of causes, chief among them being reduced renal blood flow (RBF), which causes ischemia and cell death among nephrons. Reduced RBF may be caused by cardiovascular disease, trauma, diabetes, hypertension, or reduced blood volume from dehydration. Among the many causes of kidney injury through low renal blood flow is cardiac surgery. Each year in the US, about 500,000 cardiac operations are performed, and of those, about 18% (or about 90,000 cases per year) result in some form of acute kidney injury (AKI). Around 2% of these patients (about 10,000 cases per year) will develop AKI that is severe enough to require hemodialysis. Other studies place the occurrence rate of acute kidney injury in cardiac surgery patients at 40%. In such applications, the measurement of kidney injury biomarkers must be careful to distinguish between cardiac and kidney tissue sources of the biomarkers, since both organs generate some of the same biomarkers, e.g., NGAL.

Like heart surgery, dehydration is another common cause of kidney injury from low RBF. Dehydration acts to decrease overall blood volume, and can be caused by several different factors, including inadequate water intake, prolonged exercise, the use of diuretic drugs, an illness (such as diarrhea or vomiting), or a severe hemorrhage. While monitoring everyone for dehydration-related kidney injury could prove impractical or undesirable, monitoring for kidney injury among members of particularly vulnerable groups could be valuable. For example, small children, the elderly, or individuals with chronic kidney disease may be particularly vulnerable to kidney injury during an illness. Similarly, a person starting or changing a diuretic drug regimen may benefit from monitoring for AKI to support early detection of resulting kidney injury. Or, an individual that has suffered a severe hemorrhage could be monitored for kidney damage during recovery.

Other causes of renal oxygen deprivation are hypoxemia from hemodialysis, poor lung function, or pre-eclampsia. For example, hypoxia during hemodialysis, where arterial blood oxygen tension (PₐO₂) typically falls 10-20 mm Hg, is a significant cause of kidney injury. This reduced PₐO₂ is not a problem for people with adequate oxygen tension, but patients with advanced stages of chronic kidney disease typically have lower than normal arterial oxygen levels, and this additional burden pushes such patients past the threshold where kidney injury results. Pre-eclampsia is a pregnancy-related condition that affects about 5-8% of pregnancies in the US (i.e., 200,000-320,000 cases per year), and can cause kidney injury through hypoxemia. Individuals suffering from pre-eclampsia have interrupted capillary blood flow to the nephrons that is a consequence of glomerular endotheliosis.

Toxicity likewise can have different causes, but chief among them are drug toxicity (including both pharmaceuticals and illegal drugs), industrial toxins, diabetic hyperglycemia, or trauma (which can cause the release of certain proteins that are toxic in high concentrations). Notably, along with ischemia, nephrotoxicity is a major cause of kidney injury in ICU patients, since such individuals typically receive intensive drug treatments while also suffering reduced physiological functions. Kidney injury rates for ICU patients in the US is reported to be between 20% and 50%, which equates to 800,000 to 2 million annual injuries. ICU patients suffering such injuries have mortality rates of 50% to 70%. See Vaidya, V., et al., “Biomarkers of acute kidney injury,” Annual Rev. Pharmacol. Toxicology, 2008; 48: 463-493. Early detection of AKI among this vulnerable group would therefore provide valuable guidance for physicians when determining appropriate medication levels.

Kidney inflammation can be caused by various autoimmune conditions, organ transplant rejection, infections, or IgA nephropathy. Additionally, other types of acute kidney injury, such as ischemia or toxicity, can cause an inflammatory response in the kidneys that can exacerbate an injury resulting from other causes. For example, inflammatory cytokines released into the kidney facilitate desquamation of nephron cells, which results in reduced GFR.

Blunt trauma and crush injuries to the kidney may manifest in different ways, and are difficult to assess based on imaging alone. Therefore, kidney injury biomarkers can provide much needed insight into the damage caused by physical trauma.

Some of the candidate kidney biomarkers that are measurable in sweat, and which may prove useful in assessing kidney function and injury, include NGAL, interleukin 18 (IL-18), β₂-microglobulin (B2M), α₁-microglobulin (A1M), retinol binding protein (RBP4), microalbumin (also albumin), liver-type fatty acid binding protein (L-FABP1), osteopontin, nidogen-1, cystatin C, and urea. NGAL is currently the most promising of these potential biomarkers for use in a sweat sensing device. NGAL is a 25 kDa protein that normally exists in very small amounts in the kidney tubules. However, upon the occurrence of kidney injury from ischemia or toxicity, genes coding for NGAL production are the most upregulated genes within the kidney tubules. Studies indicate that plasma NGAL levels change from a baseline level of about 22 ng/mL to approximately 145 ng/mL (7×) within 1 to 3 hours of the occurrence of kidney injury, while urinary NGAL increases from about 22 ng/mL to 560 ng/mL (25×), in the same period. See Mori, K., et al., “Endocytic delivery of lipocalin-siderophore-iron complex rescues the kidney from ischemia-perfusion injury,” J. Clin. Investigation, 115:610-621 (2005). This large, positive increase in NGAL concentration represents a physiological reaction that should facilitate NGAL's use as a kidney biomarker in sweat sensing devices. NGAL may also inform whether kidney injury is due to sepsis, since studies have indicated that NGAL is 80% higher in septic patients with kidney injury. See Martensson, C. R., et al., “Novel biomarkers of acute kidney injury and failure: clinical applicability, British Journal of Anaesthesia, Oct. 9, 2012, p. 4. An additional study has determined that urinary NGAL is a sequential predictive biomarker for AKI and is correlated with disease severity and clinical outcomes after pediatric cardiopulmonary bypass. The presence of NGAL, and in particular, its combination with IL-18, L-FABP, and/or Kidney Injury Molecule 1 (KIM-1), may help establish the timing of injury and allow earlier intervention in AKI. See Krawczeski, C. D., et al., “Temporal Relationship and Predictive Value of Urinary Acute Kidney Injury Biomarkers After Pediatric Cardiopulmonary Bypass,” J. Am. Coll. Cardiol. (2011), 58, 2301-2309. Since NGAL is known to appear in sweat, it is also likely to experience a comparable increase in sweat concentration upon the occurrence of a kidney injury.

Another candidate kidney biomarker for use in a sweat sensing device is IL-18. IL-18 is a small (18 kDa) inflammatory cytokine that is produced by renal tubules, and which plays a role in mediating several different kidney disease processes, including cancer, inflammation, ischemia, and apoptosis. IL-18 plasma concentration levels have emerged as a promising predictor for an individual to require dialysis, and for kidney injury accompanying cardiac surgery. IL-18 is present in sweat, and is likely to experience a similar change in sweat concentrations as it does in plasma in response to a kidney injury.

Cystatin C is a 13 kDa proteinase inhibitor that builds up in urine as kidney proximal tubule reabsorption capabilities are degraded. Studies indicate that cystatin C may indicate that an individual has existing kidney disease, and elevated urine levels of cystatin C may indicate increased likelihood for dialysis among ICU patients, or the presence of sepsis. Cystatin C is present in sweat and has the potential to show a similar build up in sweat in response to the existence of kidney disease.

β₂-microglobulin is a small (11 kDa) protein associated with the outer membrane of many cells including lymphocytes. It is the small subunit of the MHC class I molecule. B2M gradually accumulates in dialysis patients, and it has been implicated in the pathogenesis of amyloidosis in long-term dialysis patients. In patients with chronic renal failure, B2M levels parallel the increase in serum creatinine. Elevated levels of B2M in the blood have also been correlated with a larger tumor mass and reduced kidney function in multiple myeloma patients. The level of B2M in the blood stream is used in combination with albumin in staging multiple myeloma patients, with higher B2M levels corresponding with more advanced disease states. Although a serum protein, B2M has also been found in measurable quantities in sweat, which could enable its use as a biomarker for chronic renal disease and multiple myeloma.

α₁-microglobulin is a low-molecular-weight (26 kDa) member of the lipocalin protein superfamily. A1M is synthesized in the liver, freely filtered by glomeruli, and reabsorbed by renal proximal tubules cells, where it is catabolized. Due to this reabsorption, under healthy conditions very little filtered A1M appears in the final excreted urine. Proximal tubular function can be evaluated by measuring the amount of low molecular weight (LMW) proteins, such as A1M present in urine, since a compromised reabsorption by the proximal tubules will result in increased excretion. A1M has been detected in sweat, and it is believed that similar changes in A1M sweat concentrations levels may function as an indicator of tubular abnormalities and chronic asymptomatic renal tubular dysfunction.

Retinol binding protein is a low-molecular-weight protein (21 kDa) that transports retinol (vitamin A alcohol) from the liver to peripheral tissues. RBP4 is most often found bound to transthyretin, but a small, unbound fraction (<10%) passes freely through glomerular membranes and is reabsorbed by renal proximal tubules cells where it is catabolized. As with A1M, studies have shown that an increase in the urinary excretion of RBP4 indicates proximal tubule injury and/or impaired proximal tubular function, thereby making the measurement of RBP4 in urine a useful aid in the monitoring and/or diagnosis of kidney disease. RBP4 is present in sweat, and elevated levels in sweat may potentially serve, along with A1M, as an indicator of proximal tubular injury or dysfunction.

Microalbumin (also Albumin) is the most abundant protein found in human blood plasma and has a molecular mass of 66.5 kDa. Studies have found that lower serum albumin levels are an independent predictor of acute kidney injury, as well as death after the development of acute kidney injury. Hypoalbuminemia can be caused by kidney damage, and is common in end-stage kidney disease, as well as number of other acute and chronic medical conditions. Albumin is known to be present in sweat. However, the number of conditions that can trigger hypoalbuminemia may complicate its use as a sweat biomarker for kidney function absent other co-indicating biomarkers.

Liver-type fatty acid binding protein is a small, highly conserved, cytoplasmic protein that binds long-chain fatty acids and other hydrophobic ligands. L-FABP1 is expressed in the proximal tubules of the human kidney and participates in fatty acid metabolism. In studies, increased urinary L-FABP1 levels were found to be associated with the progression of diabetic kidney disease. The results indicated that urinary L-FABP1 levels accurately reflect the severity of diabetic kidney disease, and could serve as a clinical marker to identify patients who are likely to experience disease progression. Certain liver-type fatty acid binding proteins have been found in sweat, and others are believed to be present in measurable concentrations.

Osteopontin (OPN) is a secreted glycoprotein in both phosphorylated and non-phosphorylated forms. It contains an Arg-Gly-Asp cell-binding sequence and a thrombin-cleavage site. OPN is mainly present in the loop of Henle and distal nephrons in normal kidneys and is strongly expressed by the thick ascending limb of the loops of Henle. After renal damage, OPN expression may be significantly up-regulated in all tubule segments and glomeruli. In cases that exhibit foci of interstitial fibrosis and an associated influx of interstitial macrophages, OPN expression is significantly upregulated in all tubular segments, including proximal tubules. OPN has been detected at low levels in sweat and is a candidate sweat biomarker for indicating kidney damage or injury.

Nidogen-1 (also Entactin or Enactin) is a 150 kDa sulfated monomeric glycoprotein that serves as a linking component in basement membranes, where it interacts with laminins, collagen type IV, and proteoglycan family members. Nidogen-1, as well as Nidogen-2, have been shown to play a crucial role during organogenesis in late embryonic development, particularly in cardiac and lung development, and are believed to have a role in cell-extracellular matrix interactions. Nidogen-1 has been detected in sweat.

In addition to the above candidate kidney biomarkers which have been found in sweat, additional biomarkers indicative of kidney function when measured in blood are also believed to be present in sweat. A first of these biomarkers, Kidney Injury Molecule 1, is a 90 kDa cell membrane glycoprotein that is thought to be involved in kidney cell regeneration after an injury. KIM-1 has been shown at increased levels in urine after a kidney injury. KIM-1 RNA concentrations increase more than any other known gene after kidney injury. KIM-1 may be especially informative for toxicity, including cadmium toxicity.

Another promising biomarker that is believed to be present in measurable concentrations in sweat is N-acetyl-β-D-glucosaminidase (NAG). NAG is a 130 kDa enzyme that is involved in the hydrolysis of glucose residues in glycoproteins. NAG's concentration in urine results when nephron cells desquamate, and has been correlated with a number of kidney conditions, including established kidney disease. See Martensson, C. R., et al., p. 5. However, NAG also appears in urine for non-kidney related conditions including rheumatoid arthritis, glucose intolerance, and hyperthyroidism, which could complicate its use as a kidney biomarker for individuals with such conditions. See Vaidya, V., et al.

Uremic retention solutes are compounds that accumulate in the bloodstream when kidney function is compromised. Among this group, the molecules that exert toxicity are referred to as uremic toxins. More than 150 uremic retention solutes have been identified, and metabolomic strategies are continuing to expand this collection. Several uremic toxins have been extensively studied, but most remain to be defined and explored in the context of diagnosis and disease management. Uremic retention solutes accumulate at various rates and can reach large fold-changes with increasing loss of kidney function. Several uremic retention solutes have been identified that increase by 40- to 60-fold with uremia, and a handful of molecules can reach >100-fold changes with advanced kidney failure or genetic disorders.

Many uremic retention solutes are products of human metabolism (e.g., urea and creatinine), or of regulatory and inflammatory processes (e.g. parathyroid hormone and IL-8). See Duranton, F., et al., “Normal and Pathologic Concentrations of Uremic Toxins,” J. Am. Soc. Nephrol., 23, 1258-1270 (2010); DataBase—Eutox—European Uremic Toxin (EUTox) Work Group of the ESAO. Increased levels of these molecules may worsen pathology, or they may simply reflect the body's attempts to repair tissue or maintain homeostasis. A particularly interesting group of uremic toxins are those that originate from microbial metabolism in the gut. These uremic toxins tend to have low baseline values in healthy people, and many are of small molecular size. See Duranton, F., et al. These characteristics lend value for sweat-based monitoring, where transport into sweat, potential for rapid kinetics, and substantial fold-changes relative to baseline are important factors.

Uremic retention solutes are categorized into three groups based on their molecular properties: i) the “small molecules” (water-soluble, unbound, and <500 Da); ii) the “middle molecules” (tripeptides, cytokines, and proteins; >500 Da and as large as 50 kDa), and iii) the “protein-bound solutes” (metabolites <500 Da that bind to plasma proteins to varying degrees). See Duranton, F., et al. and Gryp, T., et al., “p-Cresyl Sulfate,” Toxins (Basel) 9, 52 (2017). Among the known uremic retention solutes, those showing the highest fold-changes in concentration levels represent the greatest potential as kidney biomarkers. For the “small molecules” group, Guanidino succinic acid (175 Da) with a 48× fold-change, and Oxalate (90 Da) with a 13× fold-change show the greatest potential as biomarkers. In the “middle molecules” group, β₂-microglobulin (11 kDa; >15× fold-change), discussed above, has the highest fold-change and, therefore, potential as a kidney biomarker. In the “protein-bound solutes” group, Hippuric acid total (179 Da; >23× fold change), Indoxyl sulfate, total (212 Da; >43× fold change), and p-Cresyl sulfate, free (>32× fold change) have the highest fold-changes in concentration levels. Of the above listed biomarkers, Oxalate, β₂-microglobulin, and Indoxyl sulfate have been detected in sweat.

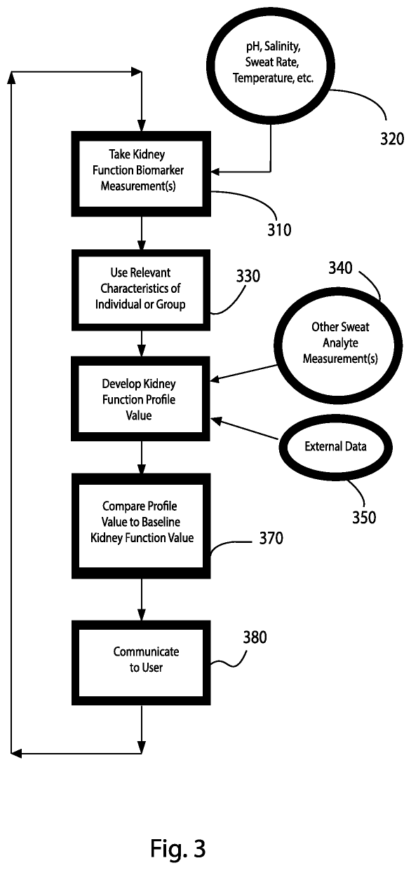

With reference to FIG. 3, to use kidney biomarker concentrations in sweat to indicate or diagnose kidney-related health conditions, a sweat sensing system 10 can be configured to develop a kidney function profile. This profile can be individualized and based on sweat analyte concentration measurements and one or more external factors, such as the factors identified above, aggregated over a period of time for a specific individual. Alternatively, reference baseline profiles can be developed for different phenotypes or groups, using sweat concentration measurements collected over a period of time from multiple individuals possessing characteristics common to the phenotype. Whether individualized or for a group, a kidney function baseline profile will incorporate sweat concentrations, trends, and ratios of kidney biomarkers, as well as additional sensor data, and will be correlated with aggregated data that is relevant to the degree of functionality demonstrated by the kidneys. Such kidney biomarkers could include, for example, NGAL, interleukin 18, β₂-microglobulin, α₁-microglobulin, retinol binding protein, microalbumin, liver-type fatty acid binding protein, osteopontin, nidogen-1, cystatin C, or urea.

To use a sweat sensing device 100 to assess the kidney function of an individual, the device measures the concentrations of one or more targeted biomarkers in a sweat sample using analyte-specific sensors 222, 224, as indicated at step 310. In addition to the targeted analytes, the device 100 may also measure additional factors relevant to kidney function or diagnosis, such as, for example, pH, salinity, sweat rate and temperature, using sensors 226, 228, as indicated at step 320. Additional data about relevant personal or group characteristics can be collected as indicated at step 330. The relevant characteristic data can be input through device 100, or downloaded or input to reader device 150, or another relevant electronic device. In addition to kidney biomarkers, other sweat analytes may be measured by the system 10, as indicated at step 340. Additionally, other external data useful in evaluating the physiological state of the individual, including, e.g., age, time of day, general health, and other factors, may be input to the system 10 as indicated at step 350.

The sweat sensor data and other secondary sensor and external data is aggregated by sweat sensing device 100, and may be used in executing one or more algorithms depending upon the processing capability of the device. Additionally, this real-time data can be communicated to reader device 150 and other computing resources, including cloud-based computing resources. The data can be aggregated and correlated with other sweat sensor data, including historical data. Using the measured sensor data as well as other measured factors and external data, including historical data for the individual or individual's phenotype, one or more algorithms can evaluate the physiological state of the individual and develop a kidney function profile value, as indicated at step 360. The kidney function profile value can be compared to a previously established baseline profile (either individualized or group) to detect differences or trends between the profile and the individual's present physiological state, as indicated at step 370. If an individual's measured biomarker level exceeds one or more applicable baseline levels, one or more physiological states may be identified based on the flagged biomarker. A notification can be communicated to the individual, caretaker, or other system user to schedule appropriate therapeutic intervention, as indicated at step 380. Additional sweat samples can be collected and evaluated from the individual to develop a trend profile for the individual, which can be used to diagnose changes in the individual's physiological state. The disclosed method thus enables a kidney function profile to be used to indicate, for example, whether an individual is experiencing a particular condition, such as a decrease in kidney function, requiring an adjustment to facilitate long-term therapeutic goals, by comparing sweat concentrations, trends, or ratios of kidney function biomarkers and other sensor data to a predetermined normal or baseline kidney function profile.

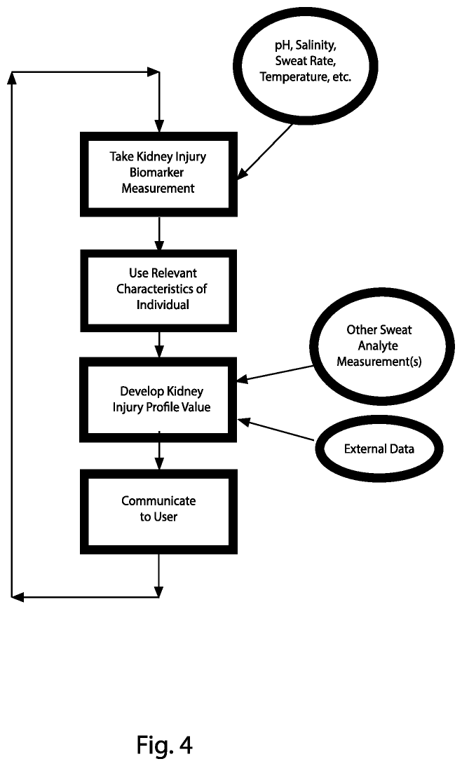

Similarly, with reference to FIG. 4, a sweat sensing device can be configured to develop and use a kidney injury profile that incorporates sweat concentrations, trends, and ratios of identified kidney biomarkers as well as additional sensor data, and can be correlated with aggregated data relevant to kidney injury. Such biomarkers could include, for example, NGAL, IL-18, or KIM-1. By comparing sweat concentrations, trends, or ratios of kidney injury biomarkers and other sensor data, the kidney injury profile could be used to indicate whether the individual likely experienced an acute kidney injury, and could further provide insight into the extent of the damage, or how recently the injury occurred.

In other embodiments, a sweat sensing device can be configured to continuously or repeatedly measure one or more kidney biomarkers in order to evaluate the need for and frequency of hemodialysis. In particular, urea is a biomarker that is known to be present in sweat and which is indicative of kidney function. The sweat sensing system disclosed herein can be configured to continuously monitor the level of urea, or other suitable biomarker present in sweat, and compare the level to a baseline profile. A baseline dialysis profile can be built for an individual through a series of measurements comparing the urea level in blood verses the urea level in sweat during similar time periods. By comparing the level of urea in sweat to the level of urea in blood, known indicator levels for dialysis can be transitioned for use with non-invasive sweat sensing. In addition to individual profiles, a number of baseline profiles for phenotypical groups can be developed. Individual sweat biomarker levels sensed by the device can then be compared to baselines for the phenotypical group to which an individual most closely identifies in order to eliminate the need to develop individual profiles. When an individual's measured biomarker level exceeds the applicable baseline, the device can notify the user to schedule dialysis. By continuously monitoring the urea level in sweat, the device wearer's dialysis can be more accurately scheduled, as current dialysis scheduling is based on a set time interval, or on discontinuous urine and blood biomarkers whose interpretation is complicated by many individual and daily factors. In another exemplary embodiment, the sweat sensing device can provide for the long-term monitoring of one or more biomarkers in a kidney transplant patient in order to optimize anti-rejection drug dosing, or to provide timely assessments of the function of the transplanted kidney.

Although not described in detail herein, other essential steps which are readily interpreted from or incorporated along with the disclosed embodiments shall be included as part of the invention. The embodiments that have been described herein provide specific examples to portray inventive steps, but will not necessarily cover all possible embodiments commonly known to those skilled in the art. Certain embodiments described herein show sensors as simple individual elements. It is understood that many sensors require two or more electrodes, reference electrodes, or additional supporting technology or features that are not captured in the description herein. Sensors are preferably electrical in nature, but may also include optical, chemical, mechanical, or other known biosensing mechanisms. Sensors can be in duplicate, triplicate, or more, to provide improved data and readings. Certain embodiments of the disclosed invention show sub-components of what would be sweat sensing devices with more sub-components needed for use of the device in various applications, which are obvious (such as a battery), and for purpose of brevity and focus on inventive aspects, are not explicitly shown in the diagrams or described in the embodiments of the present disclosure.

1. A method comprising:
receiving one or more biomarker measurements of a kidney biomarker in a sweat sample using a device configured to be worn on a skin surface of an individual;
receiving one or more secondary measurements using the device;
generating a kidney profile for the individual based on the one or more biomarker measurements and the one or more secondary measurements; and
communicating the kidney profile to a device user.

2. The method of claim 1, wherein the kidney profile is one of the following: a kidney function profile indicating a degree of kidney function of the individual; a kidney injury profile indicating whether the individual has sustained a kidney injury; a kidney injury type profile indicating a type of kidney injury the individual has sustained; a kidney injury duration profile indicating when a kidney injury event occurred; and a kidney injury severity profile indicating to what extent the individual has sustained a kidney injury.

3. (canceled)

4. (canceled)

5. The method of claim 1, wherein the one or more secondary measurements comprise one of the following: a sweat electrolyte concentration measurement, a sweat pH measurement, a sweat salinity measurement, a skin temperature measurement, a skin impedance measurement, a galvanic skin response measurement, a sweat generation rate measurement, and an accelerometry measurement.

6. The method of claim 1, wherein the one or more biomarker measurements include a measurement of a dialysis biomarker indicative of a need for hemodialysis.

7. The method of claim 6, wherein the dialysis biomarker includes at least one of urea, and a urea salt.

8. The method of claim 1, wherein the kidney profile is used to determine a physiological condition of the individual by comparing the kidney profile to: a previous kidney function profile of the individual; and a kidney function profile for a phenotypical group sharing one or more characteristics with the individual.

9. The method of claim 1, wherein the one or more biomarker measurements include a measurement of one of the following: neutrophil gelatinase-associated lipocalin, interleukin-18, kidney injury molecule-1, cystatin C, N-acetyl-β-D-glucosaminidase, β₂-microglobulin, α₁-microglobulin, retinol binding protein, microalbumin, liver-type fatty acid binding protein, osteopontin, and nidogen-1.

10. The method of claim 1, wherein the one or more biomarker measurements include a measurement of one of the following: Guanidino succinic acid; Oxalate; β₂-microglobulin; total Hippuric acid; total Indoxyl sulfate; and free p-Cresyl sulfate.

11. The method of claim 1, wherein the kidney profile includes at least one measurement of a sweat analyte that is not a kidney biomarker.

12. The method of claim 11, wherein the sweat analyte is one of the following: Na⁺, K⁺, Cl⁻, NH₄⁺, and cortisol.

13. (canceled)

14. A method comprising:
receiving one or more biomarker measurements of a kidney biomarker in a sweat sample using a device configured to be worn on a skin surface of an individual;
receiving one or more secondary measurements using the device;
generating a value representative of a physiological state of the individual by comparing the one or more biomarker measurements and the one or more secondary measurements to a baseline kidney profile; and
communicating the value representative of a physiological state of the individual to a device user.

15. (canceled)

16. The method of claim 14, further comprising comparing the value representative of a physiological state of the individual to a previous physiological state of the individual.

17. The method of claim 14, wherein the value representative of a physiological state of the individual is indicative of one or more of the following: a degree of kidney function, an occurrence of a kidney injury, a severity of the kidney injury, a type of kidney injury, and a time that the kidney injury occurred.

18. The method of claim 14, wherein the kidney biomarker is one of the following: neutrophil gelatinase-associated lipocalin, interleukin-18, kidney injury molecule-1, cystatin C, N-acetyl-β-D-glucosaminidase, β₂-microglobulin, α₁-microglobulin, retinol binding protein, microalbumin, liver-type fatty acid binding protein, osteopontin, and nidogen-1.

19. (canceled)
